(19) United States
(12) Patent Application Publication
(10) Pub. No.: US 2017/0135852 A1
(43) Pub. Date: May 18, 2017

(54) OPHTHALMIC THERMAL THERAPEUTIC APPARATUS

(71) Applicant: HUNTKEY JAPAN Inc., Tokyo (JP)

(72) Inventor: Luo CHENG, Tokyo (JP)

(21) Appl. No.: 14/940,690

(22) Filed: Nov. 13, 2015

(51) Int. Cl. A61F 7/00

(52) U.S. Cl. CPC A61F 7/007; A61F 2007/0075; A61F 2007/0004; A61F 2007/0087; A61F 2007/0071; A61F 2007/0096

(57) ABSTRACT

This disclosure provides an ophthalmic thermal therapeutic apparatus including: a handheld housing; and a thermal therapeutic apparatus provided on the housing, the thermal therapeutic apparatus including: a therapeutic member to be brought into contact with an affected area; an electrothermal conversion circuit including an electrothermal conversion element configured to generate temperature change in the therapeutic member; a temperature control apparatus configured to hold a temperature of the therapeutic member to be a desired value; a power switch; and a power source, wherein a surface of an extremity portion of the therapeutic member to be brought into contact with the affected area is formed of a convex curve having a minimum radius of curvature from 2 mm to 8 mm, preferably from 4 mm to 6 mm.

### BACKGROUND OF THE INVENTION

1. Field of the Invention

The present invention relates to heating thermal therapeutic apparatuses and heating/cooling thermal therapeutic apparatus for ophthalmic treatment (hereinafter, referred to as an ophthalmic thermal therapeutic apparatus).

2. Description of the Background Art

Heating thermal therapeutic apparatuses configured to provide treatment by warming up an affected area to promote blood circulation are widely known. Many of those, however, are of a bedclothing type or of a chair type configured to warm up the entire body, and ophthalmic therapeutic apparatuses configured to treat eye fatigue or the like have not been proposed thus far.

Portable heating thermal therapeutic apparatuses and heating/cooling thermal therapeutic apparatuses configured to locally warm up or cool down an affected area in order to treat shoulder stiffness or muscle fatigue are also known and configurations disclosed in JP-A-5-208038, JP-A-2004-223246, and JP-A-2004-305278 are known. However, none of those is ophthalmic apparatus.

Nowadays, in contrast, opportunities of watching liquid-crystal screens such as personal computers or mobile phones for a long time increase, and hence the number of people who complain eye fatigue, blurred vision, and the like increases. However, no light and effective ophthalmic thermal therapeutic apparatus is proposed.

An apparatus disclosed in JP-A-5-208038 is a heating/cooling thermal acupuncture therapeutic apparatus including a warm ball and a heater as a heat source thereof, and a cold ball and an electronic cooling element as a cold heat source thereof, which is configured to provide detection and treatment of diseases by moving the warm ball and the cold ball along a path while pushing the path. An apparatus disclosed in JP-A-2004-223246 is an electric heating thermal therapeutic apparatus for shiatsu (finger pressure therapy) or acupuncture therapy provided with a heater, an electric circuit configured to distribute electricity to the heater, a heat-transfer axis configured to transfer heat generated by the heater to a distal end of the apparatus, a rotary disc provided around the heat-transfer axis and including small projections in the periphery thereof, and a shiatsu projection provided at a distal end of the heat-transfer axis. An apparatus disclosed in JP-A-2004-305278 is a heating thermal therapeutic instrument using wood fuel as a heat source. None of the apparatuses described above are the ophthalmic thermal therapeutic apparatus.

In addition to the apparatuses described above, a heating/cooling thermal facial equipment, for example, is also proposed. However, the heating thermal therapeutic apparatus and the heating/cooling thermal therapeutic apparatus for the ophthalmic treatment have not been proposed thus far.

However, in the ophthalmologic field, therapies using warm pads or cold pads have been provided for treating eyestrain, eye fatigue, and the like, reduction of stress caused by hard use of eyes for a long time, prevention of various eye troubles.

However, these pads are consumable goods, and cost too much for long-term therapy and, in addition, have a problem of generation of waste which applies high environmental load.

If an ophthalmic thermal therapeutic apparatus which can be used in a portable manner, has high therapeutic effect, and generates no waste, especially, a heating/cooling thermal therapeutic apparatus is provided, it is quite favorable for patients suffering from eye fatigue.

### SUMMARY OF THE INVENTION

It is an object of the invention to provide an ophthalmic thermal therapeutic apparatus being compact, light, and easy to handle, and having a high therapeutic effect.

The invention is achieved by an ophthalmic thermal therapeutic apparatus including: a handheld housing; and thermal therapeutic apparatus including the following components (A) to (E) provided in the housing, that is,
(A) a therapeutic member to be brought into contact with an affected area;
(B) an electrothermal conversion circuit including an electrothermal conversion element configured to generate temperature change in the therapeutic member;
(C) a temperature control apparatus configured to hold a temperature of the therapeutic member to be a desired value;
(D) a power switch; and
(E) a power source,
   wherein an extremity portion of the therapeutic member to be brought into contact with the affected area is formed of a curved surface having a radius of curvature from 2 mm to 8 mm, preferably from 4 mm to 6 mm, especially of a rotationally symmetric convex curve.

If the radius of curvature is reduced to 2 mm or smaller, a distal end is pointed too much, and comfortable feeling of use and desirable therapeutic effect cannot be obtained, and if the radius of curvature exceeds 8 mm, the distal end of the therapeutic member cannot be brought into contact correctly with the affected area, and usage as the ophthalmic therapeutic apparatus cannot be adequately achieved. The most preferable radius of curvature is approximately 5 mm.

A main body portion of the therapeutic member has a column shape or a cylindrical shape, and the shape of the curved surface at the distal end thereof may be any one of semi-spherical, spheroidal, streamline form, egg-shaped, and slightly flattened shapes thereof, and in addition, a flat member curved into a cylindrical shape in lateral cross-sectional view.

In the case where the shape of a distal end portion of the therapeutic member is a flat shape, the radius of curvature of the extremity portion of the therapeutic member varies depending on an azimuth angle in a plane at a right angle to a central axis. However, in the invention, the minimum value of the radius of curvature falls within the above-described range, that is, from 2 mm to 8 mm, preferably, from 4 mm to 6 mm.

When the distal end of the therapeutic member has a flat shape in this manner, the apparatus fits more to the shape of the eyes which is elongated in a lateral direction, and hence a desirable therapeutic effect is obtained.

Examples of the material used for the therapeutic member include a metal such as chrome plated copper alloy or aluminum alloy. However, it is also recommended to use plastic or various composite material for achieving gentle therapeutic effect.

A thermal therapeutic apparatus of the invention is configured as a heating thermal therapeutic apparatus, cooling thermal therapeutic apparatus, or a heating/cooling thermal therapeutic apparatus.

Recommended electrothermal conversion elements are a semiconductor-type Peltier device in the case of the heating/cooling thermal therapeutic apparatus and an electrothermal heater in the case of the heating thermal therapeutic apparatus.

In the case where the Peltier device is used as the electrothermal conversion element, a heating/cooling thermal change-over switch for changing polarity of a power source is provided. In order to avoid excessive heating on a high-temperature side, it is recommended to provide a temperature control apparatus such as a temperature limit switch, and also to provide a heat exhaust apparatus such as a compact exhaust fan or a heat discharge panel. Especially, the heat exhaust apparatus is essential in the case of the heating/cooling thermal therapeutic apparatus.

The thermal therapeutic apparatus of the invention can alleviate and cure eye symptoms that many people complain nowadays at low cost easily, and thus contributes not only to an improved quality of life of general citizen, but also to a reduction of a shear of the medical bill for insurers of health cost insurance.

### BRIEF DESCRIPTION OF THE DRAWINGS

FIG. 1 is a side view illustrating an embodiment of a thermal therapeutic apparatus configured as a heating/cooling thermal therapeutic apparatus by using a Peltier device;

FIG. 2 is a front view of the thermal therapeutic apparatus illustrated in FIG. 1;

FIG. 3 is a back view of the thermal therapeutic apparatus illustrated in FIG. 1; and

FIG. 4 is a circuit drawing illustrating a configuration of an electric system of the thermal therapeutic apparatus illustrated in FIG. 1.

### DETAIL DESCRIPTION OF THE INVENTION

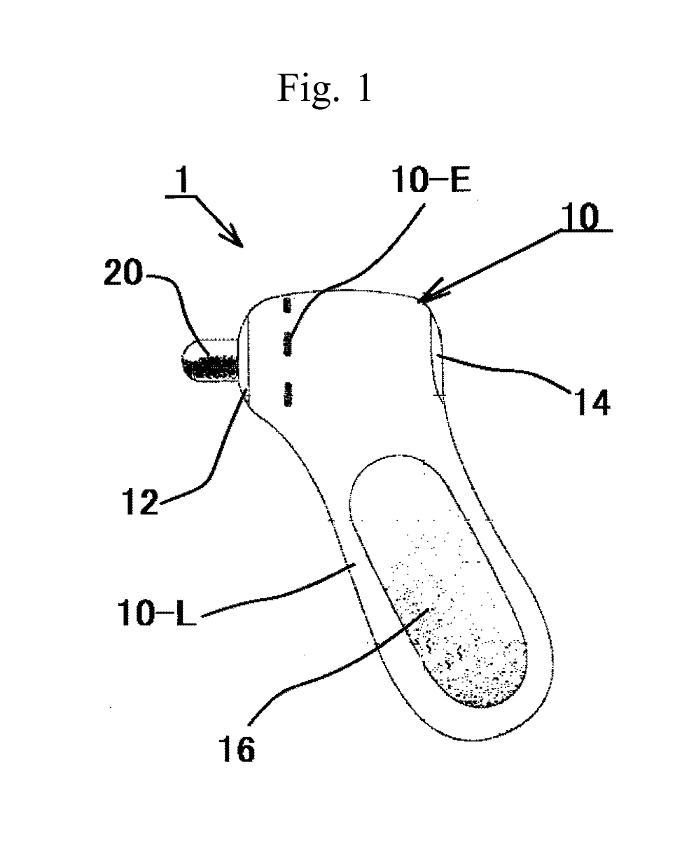

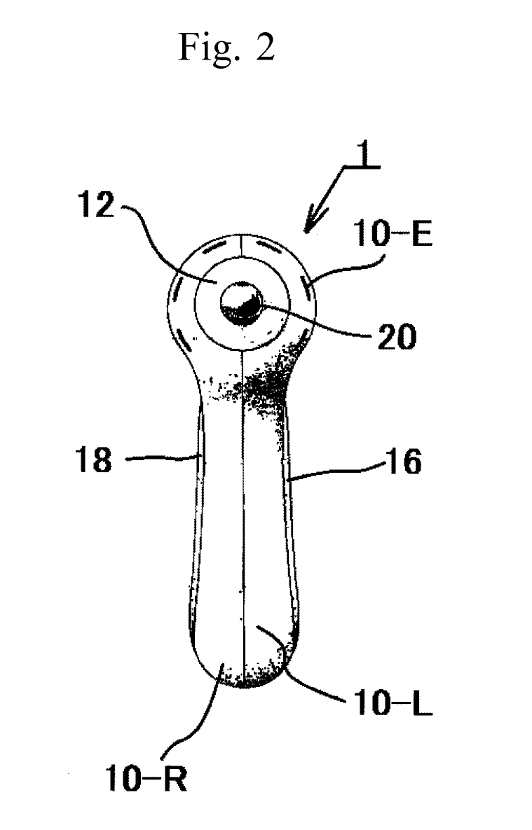

FIG. 1 to FIG. 4 are drawings illustrating a preferred embodiment of a thermal therapeutic apparatus according to the invention. In FIG. 1 to FIG. 3, reference numeral 1 denotes a heating/cooling thermal therapeutic apparatus, reference numeral 10 denotes a handheld housing, reference numeral 20 denotes a therapeutic member to be brought into contact with an affected area, reference numeral 50 denotes a power switch, and reference numeral 60 denotes a change-over switch between heating therapy and cooling therapy.

The housing 10 includes left and right shells 10-L and 10-R adhered to each other and is a hollow member formed into a substantially L-shape including a head portion and a grip portion. A thermal therapeutic apparatus 1 is provided on a front side of the head portion with a front lid member 12, on a rear side of the head portion with a rear lid member 14, and on left and right of the grip portion with left and right side lid members 16 and 18, respectively. The thermal therapeutic apparatus 1 accommodates a heat source and part of the therapeutic member 20 which is to be brought into contact with the affected area in an interior thereof.

Here, the left side lid member 16 is a lid member for a cell chamber. The right side lid member 18 is a simple lid member configured to close a void portion.

The housing 10 is provided with a plurality of front air ventilating holes 10-E on the head portion at positions biased to the front so as to surround the front portion of the head portion. The rear lid member 14 is provided with a number of large and small rear air ventilating holes 14-E concentrically in line symmetry in a radiated manner.

The therapeutic member 20 projects coaxially with the head portion of the housing 10 from the center of the front lid member 12 of the housing 10. The therapeutic member 20, which is described later, is configured to be brought into contact with an eyelid or peripheries thereof of a patient in a state of being warmed up or cooled down by a heat source or a cold heat source integrated in the housing 10 for the purpose of a therapy.

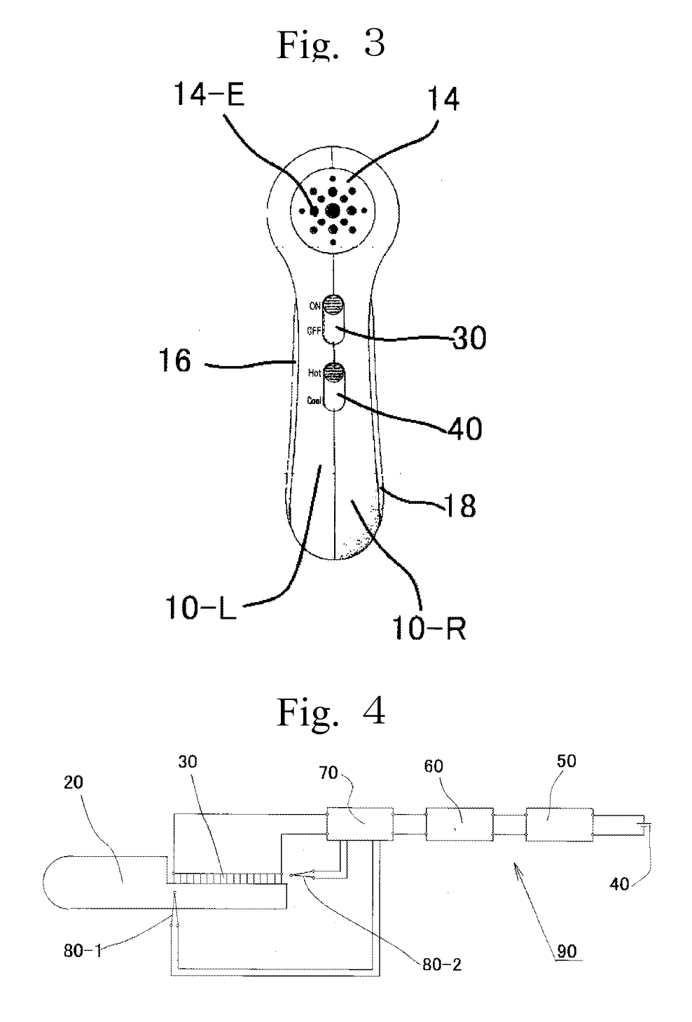

As illustrated in FIG. 3 and FIG. 4, a Peltier device 30, a power source 40, a power source switch 50, a change-over switch 60 for the heating therapy and the cooling therapy, a temperature limit switch 70, and temperature sensors 80-1 and 80-2 are provided in an interior of the housing 10. Although not illustrated, the cell chamber is formed in an interior of the grip portion, so that the power source 40 is accommodated therein.

Employed in this embodiment is a configuration in which a main body portion of the therapeutic member 20 has a column shape, and a distal end portion which projects from the housing 10 and is to be pushed against an affected area has a semi-spherical shape, and a rear end portion is machined into a semi-column shape to provide a plane on which the Peltier device 30 is mounted.

However, the shape of the therapeutic member 20 is not limited to the shapes described above. For example, the main body portion may have a rectangular column. The shape of the distal end portion which is brought into contact with the affected area is not limited to the spherical shape, and may be a spheroid or a streamline form or, in addition, those which are not a rotation symmetrical shape, that is, those having a non-circular cross section taken at a right angle with respect to the central axis, and for example, flat shapes such as a laterally ellipsoidal shape and an oval shape are also applicable.

In the case where the distal end of the therapeutic member 20 has a rotation symmetrical shape, for example, a semi-spherical shape, the radius of curvature in the invention is set to fall within a range from 2 mm to 8 mm, preferably from 4 mm to 6 mm.

In the case where the shape of the distal end portion of the therapeutic member 20 is not a rotation symmetrical shape, the radius of curvature varies depending on an azimuth angle on a plane orthogonal to the central axis of the therapeutic member 20. However, in the invention, the minimum value of the radius of curvature is set to fall within a range from 2 mm to 8 mm, preferably, from 4 mm to 6 mm.

In this shape, the distal end of the therapeutic member fits the shapes of eyepits or peripheries of the eyes of general adults, so that adequate heating therapy is enabled.

In order to reduce thermal equivalent of the therapeutic member 20, the main body portion may be formed into a circular tube shape. The therapeutic member 20 may be fixed to the housing. However, a configuration of being held so as to be movable forward and backward within a certain range via a shock absorbing member such as a coil spring is also recommended.

If a metal having a high heat conductivity such as aluminum alloy, copper, or copper alloy is used as a material of the therapeutic member 20, the semispherical portion at the distal end of the therapeutic member 20 reaches a temperature suitable for therapy in a short time when the power switch is turned ON. However, if a material having a rather low heat conductivity is used, gentle therapy is enabled, and thus a composite material having a suitable heat conductivity is also applicable.

The invention is characterized in that a surface of the therapeutic member 20 which comes into direct contact with an affected area is formed to be a convex curve having the minimum radius of curvature from 2 mm to 8 mm, preferably from 4 mm to 6 mm.

If the radius of curvature is below 2 mm, the distal end becomes pointed too much, and thus a comfortable feeling of use cannot be obtained. If the radius of curvature exceeds 8 mm, adequate usage as the ophthalmic therapeutic apparatus is impaired.

The radius of curvature which provides a desirable feeling of use is from 4mm to 6 mm, and the most desirable radius of curvature is approximately 5 mm.

FIG. 4 is an explanatory drawing illustrating a circuit configuration of a therapeutic apparatus illustrated in FIGS. 1 to 3, in which reference numeral 30 denotes the Peltier device, reference numeral 40 denotes the power source, reference numeral 50 denotes the power switch, reference numeral 60 denotes the heating/cooling change-over switch, reference numeral 70 denotes the temperature limit switch, reference numerals 80-1 and 80-2 denote the temperature sensors, and reference numeral 90 denotes a thermoelectric converting circuit including the Peltier device 30 and the heating/cooling change-over switch 60.

In this embodiment, a Peltier device mounting portion of the therapeutic member 20 is chamfered into a semi-circular shape in cross-section, and the flat-panel shaped Peltier device 30 is mounted on a flat surface portion of the therapeutic member 20. However, using a member having so-called a skeleton structure formed by arraying P-type and N-type semiconductor devices alternately on a flexible base (separator), and connecting the same in series with a copper electrode is wound around the therapeutic member 20 or adhered thereto or, alternatively, using a Peltier device formed into a cylindrical shape is also recommended.

Description of functions of the power source 40 and the power source switch 50 will not be necessary.

The heating/cooling change-over switch 60 is configured to change over the temperature of the therapeutic member 20 to either a state of high-temperature therapy, for example, between 40 and 45° C. or a state of low-temperature therapy, for example, between 10 and 20° C.

The temperature limit switch 70 is ON-OFF controlled by an output signal of the temperature sensors 80-1 or 80-2, and an electric current of the Peltier device 30 is controlled so that the temperature of a portion of the therapeutic member 20 which becomes a high temperature does not exceed a certain allowable temperature, for example, 42° C.

When using the ophthalmic thermal therapeutic apparatus of the invention, a contact point of the heating/cooling change-over switch 60 is selected and set to meet desired therapy, the power source switch 50 is turned ON, and after the therapeutic member 20 has become an adequate temperature, the patient closes his/her eye, the distal end portion of the therapeutic member 20 is brought into light contact with the eyelid or the periphery thereof to provide treatment. By using the ophthalmic thermal therapeutic apparatus, therapeutic effects such as recovering eye strain and decreasing inflammation are achieved.

According to the thermal therapeutic apparatus of the invention, since the radius of curvature of a portion of the therapeutic member 20 which comes into contact with the eyelid that covers the eye as an affected area and the peripheral edge portion thereof is formed into a convex curve having a radius of curvature falling within a range from 2 mm to 8 mm, preferably, a radius of curvature falling within a range from 4 mm to 6 mm, further preferably, a convex curve having a radius of curvature approximately 5 mm, the therapeutic member 20 is capable of coming into tight contact adequately with an affected area having a complex curved surface and thus a comfortable feeling of treatment is obtained and a high therapeutic effect is achieved.

The thermal therapeutic apparatus of the invention easily cures diseases such as eye fatigue, which is one of modern disease, from which many people are suffering, and thus contributes to an improvement of social life quality of ordinary person, and also contributes to reduction of a shear of health cost insurance.

What is claimed is:

1. An ophthalmic thermal therapeutic apparatus comprising:
a handheld housing; and
a thermal therapeutic apparatus provided on the housing, the thermal therapeutic apparatus including:
a therapeutic member configured to come into contact with an affected area;
an electrothermal conversion circuit including an electrothermal conversion element configured to generate temperature change in the therapeutic member;
a temperature control apparatus configured to hold a temperature of the therapeutic member to be a desired value;
a power switch; and
a power source,
wherein a curved surface of an extremity portion of the therapeutic member which comes into contact with the affected area is a convex curve having a minimum radius of curvature falling within a range from 2 mm to 8 mm.

2. The ophthalmic thermal therapeutic apparatus according to claim 1, wherein
a curved surface of the extremity portion of the therapeutic member which comes into contact with the affected area is a convex curve having the minimum radius of curvature falling within a range from 4 mm to 6 mm.

3. The ophthalmic thermal therapeutic apparatus according to claim 1, wherein
the electrothermal conversion element is a Peltier device, and the electrothermal conversion circuit includes a heating/cooling thermal change-over switch.

4. The ophthalmic thermal therapeutic apparatus according to claim 2, wherein
the electrothermal conversion element is a Peltier device, and the electrothermal conversion circuit includes a heating/cooling thermal change-over switch.

5. The ophthalmic thermal therapeutic apparatus according to claim 1, wherein
the electrothermal conversion element is an electric resistive element.

6. The ophthalmic thermal therapeutic apparatus according to claim 2, wherein
the electrothermal conversion element is an electric resistive element.
